(19) United States
(12) Patent Application Publication
(10) Pub. No.: US 2005/0228338 A1
(43) Pub. Date: Oct. 13, 2005

(54) Apparatus and method for transdermal delivery of fluids including oxygen

(76) Inventor: Ronald Allen Greenberg, Woodforde (AU)

Correspondence Address: WEINER & BURT, P.C., 635 N. US-23, P.O. BOX 186, HARRISVILLE, MI, 48740, US

(21) Appl. No.: 10/839,555

(22) Filed: May 5, 2004

(30) Foreign Application Priority Data
Apr. 8, 2004 (AU) AU 2004201456

(51) Int. Cl. A61M 37/00

(52) U.S. Cl. 604/23

(57) ABSTRACT

The present invention relates to an apparatus and method for delivering or infusing oxygen into the deep layers of the skin. This is achieved through firstly spraying the skin area with a pre-oxygenated fluid and then causing the solution to permeate into the skin by applying a greater than atmospheric pressure to the sprayed skin. The apparatus provides for an air compressor that draws fluid from a tank to be sprayed through a trigger nozzle onto the skin. The end of the nozzle further includes a cavity defined by a peripheral skirt that engages against the skin to seal and pressurise an area.

The present patent application is based on and claims priority from Australian Patent Application AU2004201456, filed Apr. 8, 2004.

### BACKGROUND OF THE INVENTION

Methods for revitalisation and stimulation of the skin have long been sought after, and many cosmetic techniques have been developed over the years as a result. The method of simply applying, rubbing or spraying cosmetic treatments onto the skin continues to be the most common method of skin treatment. However, it has been widely agreed upon in recent years that for skin treatments to take their full effect, they must penetrate through the epidermal layers of the skin into the deeper layers such as the dermis and hypodermis where assimilation with cells is much easier and a more thorough treatment is achieved. Newer methods of achieving transdermal delivery have therefore evolved. For example, injections of cosmetic treatments into the skin using a syringe or infusion into the skin under pressurised application are now common practices.

It is also well known that skin cells begin to die without sufficient oxygen and as human beings become older, the level of oxygen in the skin significantly decreases. Therefore, newer methods have also involved the introduction of gaseous oxygen into the skin, and are achieved through pressurised application. This typically involves the use of a hand-piece through which flows pressurised gaseous oxygen, including a nozzle which is adapted to be placed against the skin whereby the oxygen, mixed or entrained with a cosmetic solution within the hand-piece, is infused into the skin. There are however a number of drawbacks to these existing methods of transdermal delivery. These include:
Syringes in general are not a favoured treatment of patients for obvious reasons:
when using pressurised gaseous oxygen, the oxygen draws moisture from the cells resulting in skin dehydration;
pressurised gaseous oxygen does not readily assimilate with skin cells, this being a key requirement in an effective skin treatment;
the pressurised gaseous oxygen technique does not allow for existing cosmetic solutions to be sprayed onto the skin prior to pressurised application; and
existing techniques involve the use of complex machinery such as filters and separation chambers for filtering outside air to form pure oxygen.

It is therefore an object of the present invention to overcome at least some of the aforementioned problems or to provide the public with a useful alternative.

It is a further object of the present invention to provide a method of first spraying an oxygen-enriched cosmetic solution onto a skin surface and to then deliver the solution into the deep layers of the skin beyond the epidermal layer.

It is a still further object of the present invention to provide an apparatus for the treatment of skin, the apparatus including an air compressing means and an outlet for the compressed air in the form of an attached air brush or like hand-piece including a nozzle and a solution containment means. A solution, that may be oxygenated, contained within the containment means is entrained with the compressed air at the nozzle and exits the nozzle in the form of a spray. Once the solution has been applied, the nozzle is brought into a sealing effect with the skin surface to thereby infuse the cosmetic solution into the skin by pressurised air means.

### SUMMARY OF THE INVENTION

Therefore in one form of the invention there is proposed an apparatus for transdermal delivery of a fluid to skin, said apparatus including:
a source of compressed gas connected to an outlet device, said outlet device including a nozzle, a trigger, a valve and a fluid tank containing said fluid;
said valve configured to control flow of said compressed gas through said aperture and whereby said flow of air causes said fluid to spray out of said nozzle;
wherein when said skirt is caused to engage said skin to said air pressure assists in injecting at least some of said fluid into said skin.

Preferably said source of compressed gas is an air compressor.

Preferably said nozzle includes a single aperture through which said compressed gas passes.

Preferably said nozzle includes multiple apertures though which passes said compressed gas and fluid.

Preferably said fluid contains at least in part oxygen.

Preferably said valve is a trigger type valve.

In a further form of the invention there is proposed a method for transdermal delivery of oxygen to a person, said method including the steps of:
spraying a fluid including at least in part oxygen to the skin of said person;
sealingly isolating an area of said skin onto which said fluid has been sprayed and applying a greater than atmospheric pressure thereto to cause at least some of said fluid to permeates into said skin.

### BRIEF DESCRIPTION OF THE DRAWINGS

The accompanying drawings, which are incorporated in and constitute a part of this specification, illustrate several implementations of the invention and together with the description, serve to explain the advantages and principles of the invention. In the drawings,

FIG. 1 is a perspective view of the apparatus embodying the present invention for the transdermal delivery of a fluid;

FIG. 2 is a cross-sectional view of human skin illustrating the penetration of substances applied using various methods of treatment described in the prior art:

FIG. 3 is a perspective view of the handheld outlet device of the present invention illustrating with broken lines the internal duct through which the pressurised atmospheric air passes; and

FIG. 4 is a cross-sectional view of human skin illustrating the penetration of the cosmetic solution applied according to the present invention.

### DESCRIPTION OF THE PREFERRED EMBODIMENTS

The following detailed description of the invention refers to the accompanying drawings. Although the description includes exemplary embodiments, other embodiments are possible, and changes may be made to the embodiments described without departing from the spirit and scope of the invention. Wherever possible, the same reference numbers will be used throughout the drawings and the following description to refer to the same and like parts.

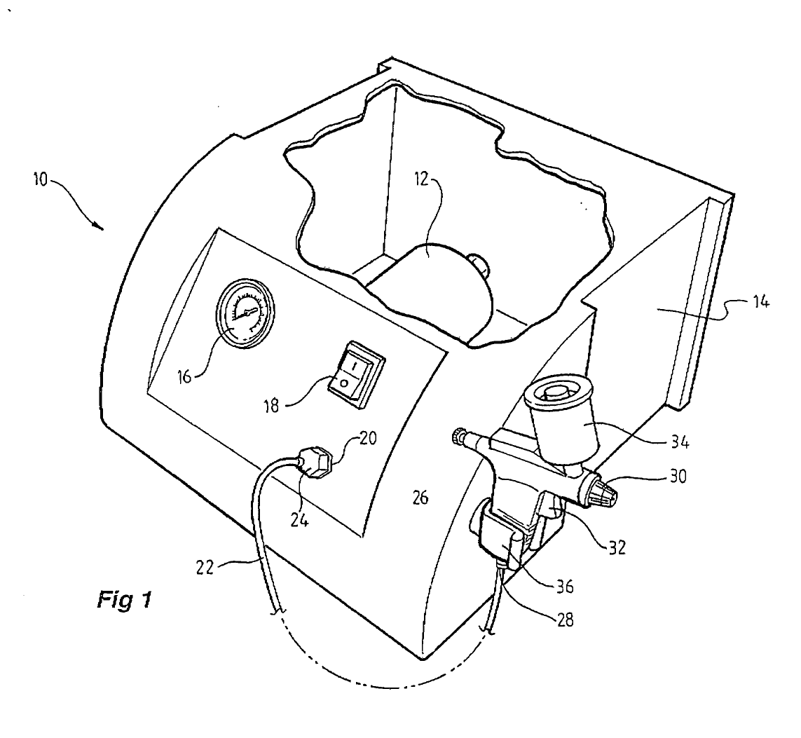

Referring to FIG. 1 there is illustrated an apparatus 10 for the transdermal delivery of a fluid containing at least in part oxygen, including, an air compressor 12, mounted within case 14, pressure gauge 16, power control switch 18 and an outlet 20. Tube 22 is connected to the outlet 20 of the air compressor 12 via connection means or coupling 24. The opposite end of the tube 22 is connected to a handheld outlet device 26 via connection means or second coupling 28. The handheld outlet device 26 includes a nozzle 30, a trigger 32 and a tank 34, and is in pressurised communication with the air compressor 12 through tube 22. The configuration of the handheld outlet device 26 can be in the form of a pistol grip device. However it should be understood by the reader that the present invention is not restricted to this configuration and any handheld device, which facilitates ease of use, could be used. The apparatus 10 can also incorporate other features such as a bracket 36 for holding the outlet device 26 or a clock (not shown) to measure the duration of treatment (not shown).

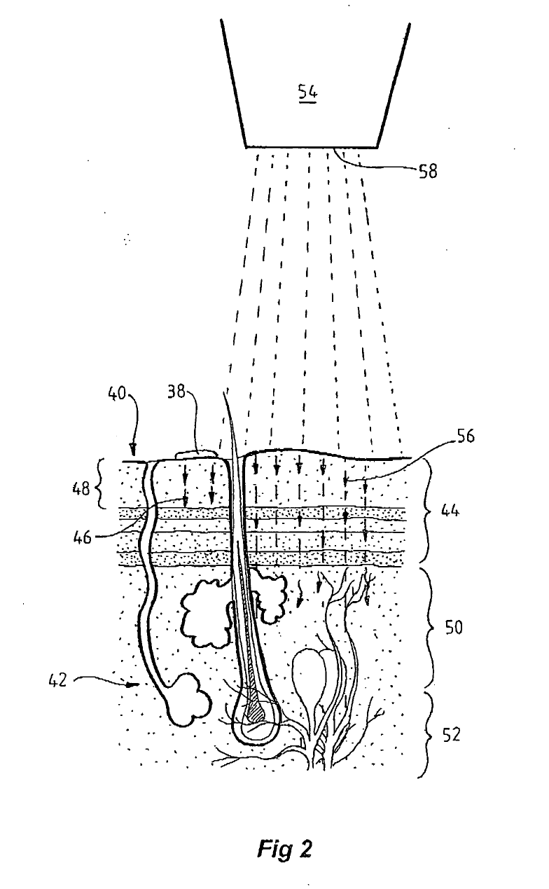

The treatment of human skin for therapeutic and aesthetic reasons has occurred throughout the centuries. Many of these methods involved the application of cosmetic lotions or creams 38 to the outer surface 40 of skin 42 as illustrated in FIG. 2. The lotion or cream 38 is typically rubbed into the surface of the skin 40 and is allowed to diffuse into the epidermal layer 44. This type of treatment however has a distinct drawback as the lotion or cream 38 usually only penetrates a short distance (46) into the stratum corneum 48 of the epidermal layer 44.

But, it is known that deeper penetration lotions or creams increases their efficiency. This has led to various techniques being developed to increase the level of penetration. For example, syringes have been used to convey cosmetics directly to the deeper layers of skin. The use of needles however causes anxiety and fear in some patients. Furthermore, with the advent of AIDS many people shy away from unnecessary use of needles. Consequently, other methods have been developed to deliver cosmetics into the deeper layers of the skin such as the dermis 50 and hypodermis 52.

Oxygen is well known for the role it plays in the health and development of aerobic cells. Oxygen enhances the body's ability to metabolise, absorb, assimilate and utilise vitamins, minerals and other nutrients. As skin ages it looses its ability to consume and retain oxygen. It is believed that reduced oxygen is one of the causes of wrinkles, dark circles, puffy eyes and dull looking skin. Oxygen increases the metabolic health of cells, enhances collagen production and improves the functioning of blood vessels. Consequently, oxygen treatments are used in the cosmetic industry to overcome the visible effects of aging. Various methods have been developed over the years to utilise oxygen for the therapeutic treatment of human skin, including oxygen enhanced creams and lotions that are widely available. These however have the disadvantage of only penetrating the surface layer of the skin. Therefore, other methods have been developed for the delivery of oxygen-enhanced substances into the dermal 50 and hypodermal 52 layers of skin.

One current method introduces gaseous oxygen into the skin 42 by pressurised application. As illustrated in FIG. 2 an apparatus 54 is configured to deliver pressurised gaseous oxygen into the skin 42. The pressurised oxygen penetrates (56) the epidermis 44 thereby making oxygen available in the deeper layers of skin 50 and 52. There are however a number of disadvantages with this type of application. Pressurised gaseous oxygen draws moisture away from cells that can produce toxic results. Gaseous oxygen is also not readily bio-available to cells. Furthermore, the oxygen used in this method is very unstable and can produce harmful free radicals.

To overcome some of these problems such methods use stabilisers and precursors to control the reactive nature of the oxygen. This then creates other problems, as these stabilisers can be carcinogenic. The treatment area of such methods is also reduced. Another problem with this system is that the equipment requires a series of filters and pumps that purify atmospheric air into an oxygen rich mixture. This configuration increases the cost of production and maintenance of the equipment.

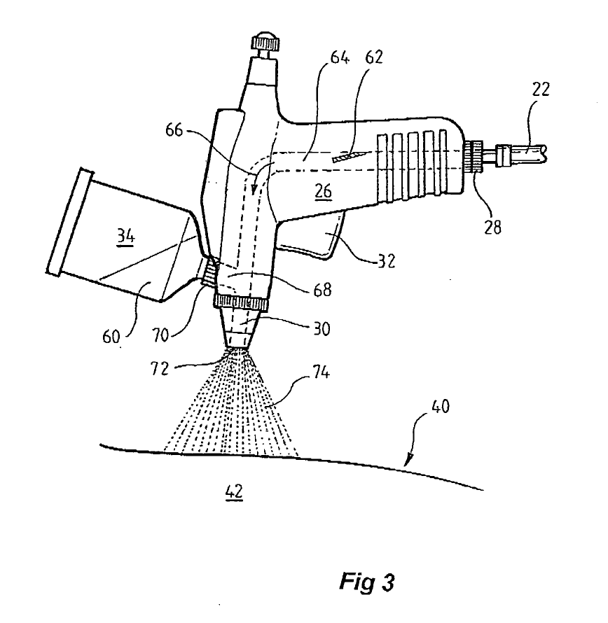

The present invention provides an apparatus and method of delivering an oxygen-enriched substance to the deeper layers of the skin without the use of pressurised gaseous oxygen. This avoids the potential toxic effects that can be caused by such use. In a first step, as illustrated in FIG. 3, the oxygen enriched fluid or cosmetic solution 60 is sprayed onto the skin surface 40 by means of a handheld outlet device 26 that is connected to an air compressor 12 via tube 22. The outlet device 26 incorporates a valve 62 that is configured to control the flow of pressurised air through the nozzle 30. As the trigger 32 is depressed the valve 62 moves from a first position wherein pressurised air is not allowed to pass to a second position wherein pressurised air is allowed to flow through the internal duct 64 of the outlet device 26 in the direction of arrow 66.

The cosmetic solution 60 that contains oxygen is accommodated within the containment means or tank 34, which is connected to the outlet device 26 by connection means or coupler 70. The connection means 70 couples with outlet 68 to ensure that there is no leakage of the fluid or cosmetic solution 60 during use. It should however be understood by the reader that the tank or containment means 34 is not confined to that illustrated in FIG. 3. The configuration and position of the containment means is not essential to the invention as any type of refillable container that can be pressurised could be used. In preference the container can be removed although a permanent compartment within the outlet device 26 could alternatively be used to accommodate the fluid or cosmetic solution 60. The cosmetic solution 60 can also contain therapeutic agents. These therapeutic agents can be selected from any substance that has beneficial properties and can include minerals such as calcium, magnesium, phosphorus, sulphur, selenium, silica and copper or enzymes such as CoQ10.

As the pressurised air passes the opening 68 the cosmetic solution 60 accommodated within the containment means 34 is entrained by said pressurised air flowing in the direction of arrow 66 through the nozzle 30 through the well known Venturi effect. The pressurised air now including at least in part the cosmetic solution in suspension then exits the outlet device 26 through aperture 72 in the form of a spray 74. In this way the cosmetic solution 60 is sprayed 74 onto the skin surface 40 of the patient. The distance between the outlet device 26 and the skin surface 40 will depend on the area that is being treated. However, to effectively cover a treatment area the outlet device 26 should be held in close proximity to the treatment area. This distance may be desirably very small if the treatment area is only of a limited size or if the treatment area is close to an orifice such as the nose or ears.

The aperture 72 of the nozzle 30 is preferably a single outlet. Alternatively a series of smaller apertures similar to those of a shower rosette could be used. A filter (not shown) could be used to remove any impurities in the cosmetic solution 60. Accessories (not shown) could also be used to direct the pressurised flow of air. These accessories could be removably attached to the nozzle and thereby constrain the flow of air to a particular profile. This could be useful when specific areas such as the area around the eyes require treatment.

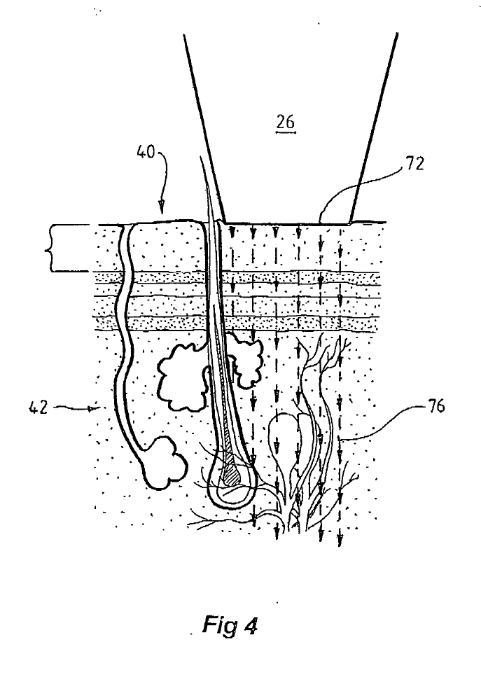

Once the cosmetic solution 60 has been sprayed (74) onto the skin surface 40, the aperture 72 of the outlet device 26 is brought into very close proximity or in contact to the skin surface 40 as illustrated in FIG. 4. There the outer skirt 71 of the nozzle 30 sealingly engages an area of skin creating a pressurised chamber. In this position the pressure differential between the chamber and the skin 42 causes at least some of the fluid to forcibly permeate into the skin that by its own structure is quite porous. The cosmetic solution thus invades (76) the deeper layers of the skin 42 rather than slowly and unreliably infusing. In this way the oxygen and other therapeutic agents contained within the cosmetic solution 60 are more readily and controllably deposited in a location where they can be utilised by the cells in the deeper layers of the skin 42. After the chamber has been effectively pressurised, the flow of air and thus solution is reduced or even stopped until the tool is removed away from the skin when the solution can be appropriately sprayed again.

In one aspect the tank or fluid containment means 34 is filled with only enough cosmetic solution 60 to cover one treatment area of skin surface 40. Once all of the cosmetic solution 60 has been sprayed onto the skin surface 40 and the containment means 34 is empty, any further depression of the trigger 32 will only result in pressurised air flowing out through the nozzle 30 of the outlet device 26. The user then rests the nozzle against the skin where, even though no fluid flow may occur there is a pressure differential.

In a further aspect of the invention the containment tank 34 is filled with enough cosmetic solution 60 to cover several treatment area of skin surface 40. The cosmetic solution 60 is sprayed onto the skin surface 40 in a manner discussed earlier, so that the against the skin surface 40. The trigger is then depressed to allow the nozzle to be pressurised thereby entraining the cosmetic solution 60 into the deeper layers of the skin 42. The pressurised air that emanates from the nozzle 30 may contain a proportion of cosmetic solution 60 if indeed there is leakage form the nozzle. With a good seal however, there is no fluid flow and no further fluid is discharged onto the skin resulting in reduced wastage of the solution.

As the reader would now appreciate the present invention provides a useful method and apparatus for delivering oxygen enriched cosmetic solution into the deep layers of the skin beyond the epidermal layer. The present method does not use gaseous oxygen and therefore avoids its corresponding toxic effects. It also does not use substances containing hydrogen peroxide, chlorate or chlorite oxygen that can also have toxic consequences. The ability to safely force cosmetic solutions into the deeper layers of the skin without the use of needle is also a significant advantage. Oxygen treatments are popular in cosmetic industry today and the present invention provides the public with a method and apparatus that overcomes the limitations of the currently available methods. This non-toxic, non-invasive method of inserting cosmetic solutions containing oxygen and other therapeutic agents into the skin has significant benefits over these currently available methods.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus.

1. An apparatus for transdermal delivery of a fluid to skin, said apparatus including:
a source of compressed gas connected to an outlet device, said outlet device including a nozzle defining a chamber, a trigger, a valve and a fluid tank containing said fluid;
said valve configured to control flow of said compressed gas through said aperture and whereby said flow of air causes said fluid to spray out of said nozzle;
wherein when said skirt is caused to engage said skin said compressed gas pressurizes said chamber assisting in forcibly injecting at least some of said fluid into said skin.

2. An apparatus as claimed in claim 1, wherein said source of compressed gas is an air compressor.

3. An apparatus as in claim 1, wherein said nozzle includes an aperture through which said compressed gas passes, said aperture being located within said chamber.

4. An apparatus as in claim 2, wherein said nozzle includes an aperture through which said compressed gas passes, said aperture being located within said chamber.

5. An apparatus as in claim 1, wherein said nozzle includes multiple apertures through which passes said compressed gas and fluid.

6. An apparatus as in claim 2, wherein said nozzle includes multiple apertures through which passes said compressed gas and fluid.

7. An apparatus as in claim 3, wherein said nozzle includes multiple apertures through which passes said compressed gas and fluid.

8. An apparatus as in claim 4, wherein said nozzle includes multiple apertures through which passes said compressed gas and fluid.

9. An apparatus as in claim 1, wherein said fluid contains at least in part oxygen.

10. An apparatus as in claim 2, wherein said fluid contains at least in part oxygen.

11. An apparatus as in claim 3, wherein said fluid contains at least in part oxygen.

12. An apparatus as in claim 4, wherein said fluid contains at least in part oxygen.

13. An apparatus as in claim 5, wherein said fluid contains at least in part oxygen.

14. An apparatus as in claim 6, wherein said fluid contains at least in part oxygen.

15. An apparatus as in claim 7, wherein said fluid contains at least in part oxygen.

16. An apparatus as in claim 8, wherein said fluid contains at least in part oxygen.

17. An apparatus as in claim 1, wherein said valve is a trigger type valve.

18. An apparatus as in claim 2, wherein said valve is a trigger type valve.

19. An apparatus as in claim 3, wherein said valve is a trigger type valve.

20. A method for transdermal delivery of oxygen to a person, said method including the steps of:
spraying a fluid including at least in part oxygen to the skin of said person;
sealingly isolating an area of said skin onto which said fluid has been sprayed and applying a greater than atmospheric pressure thereto to cause at least some of said fluid to permeate into said skin.
